(19) United States
(12) Patent Application Publication
(10) Pub. No.: US 2026/0041420 A1
(43) Pub. Date: Feb. 12, 2026

(54) SUTURING DEVICE WITH NEEDLE CATCH

(71) Applicant: Boston Scientific Scimed, Inc., Maple Grove, MN (US)

(72) Inventor: Luis Parra, Barrio San Jose (CR)

(21) Appl. No.: 19/291,111

(22) Filed: Aug. 5, 2025

Related U.S. Application Data
(60) Provisional application No. 63/681,602, filed on Aug. 9, 2024.

(51) Int. Cl. A61B 17/04

(52) U.S. Cl. CPC A61B 17/0491

(57) ABSTRACT

The invention relates to a suturing device used to please sutures inside of a body of a patient during various medical procedures. The apparatus includes an elongate member, a needle configured to extend from a portion of the elongate member, and a needle catch configured to receive at least a portion of the needle The needle catch has a plate member defining an opening. The plate member has an edge portion that is disposed adjacent to the opening. The edge portion of the plate member has a curved portion. The curved portion is curved about a center of curvature. At least a portion of the opening being disposed between the edge portion and the center of curvature.

### CROSS-REFERENCE TO RELATED APPLICATION

This application claims priority to U.S. Provisional Patent Application No. 63/681,602, filed on Aug. 9, 2024, entitled “SUTURING DEVICE WITH NEEDLE CATCH”, the disclosure of which is incorporated by reference herein in its entirety.

### TECHNICAL FIELD

The present invention generally relates to medical devices, particularly devices that include a needle and a needle catch or receiving portion.

### BACKGROUND

Suturing of body tissues or suturing bodily implants to bodily tissue may be time consuming and an important aspect of some surgical procedures. In some cases, suturing devices are used for such surgical procedures. So suturing devices may be placed within the body and actuated to move a needle within the body pass the needle through bodily tissue and/or bodily implants. In some cases, such suturing devices may be used to attach our couple the bodily implants to the bodily tissue.

In some cases, the suturing devices include a needle catch that is configured to receive a portion of the needle after it has passed through bodily tissue and/or a bodily implant. Therefore, there is a need for catch members or portions that effectively and reliably catch and secure the needle after the needle has passed through bodily tissue and/or a bodily implant.

### SUMMARY

According to an aspect, an apparatus or a suturing device used to please sutures inside of a body of a patient during various medical procedures. The apparatus includes an elongate member, a needle configured to extend from a portion of the elongate member, and a needle catch configured to receive at least a portion of the needle. The needle catch has a plate member defining an opening. The plate member has an edge portion that is disposed adjacent to the opening. The edge portion of the plate member has a curved portion. The curved portion is curved about a center of curvature. At least a portion of the opening being disposed between the edge portion and the center of curvature.

In some implementations, the edge portion of the plate member is a first edge portion of the plate member, wherein the plate member includes a second edge portion that is disposed adjacent the opening, the second edge portion of the plate member having a curved portion. In some implementations, the edge portion of the plate member being a first edge portion of the plate member, wherein the plate member includes a second edge portion that is disposed adjacent the opening, the second edge portion of the plate member having a curved portion, the second edge portion being disposed on a first side of the opening defined by the plate member, the second edge portion being disposed on a second side of the opening defined by the plate member. In some implementations, the edge portion of the plate member being a first edge portion of the plate member and the center of curvature being a first center of curvature, wherein the plate member includes a second edge portion that is disposed adjacent the opening, the second edge portion of the plate member having a curved portion, the curved portion of the second edge portion being curved about a second center of curvature, at least a portion of the opening being disposed between the second edge portion and the second center of curvature.

In some implementations, the plate member is non-planar. In some implementations, a portion of the plate member disposed adjacent to the edge portion is non-planar.

In some implementations, the opening defined by the plate member includes a first slot portion and a second slot portion, the edge portion being disposed between the first slot portion and the second slot portion. In some implementations, the opening defined by the plate member includes at least 5 slot portions. In some implementations, the opening defined by the plate member includes a first slot portion, a second slot portion, a third slot portion, a fourth slot portion, and a fifth slot portion.

In some implementations, the opening defined by the plate member is a first opening, the plate member defining a second opening. In some implementations, the opening defined by the plate member is a first opening, the plate member defining a second opening and a third opening, the first opening being disposed between the second opening and the third opening.

In some implementations, the curved portion of the edge portion of the plate member is a first curved portion, wherein, the edge portion of the plate member has a second curved portion. In some implementations, the curved portion of the edge portion of the plate member is a first curved portion, wherein, the edge portion of the plate member has a second curved portion, the second curved portion of the edge portion having a center of curvature, at least a portion of the opening defined by the plate member being disposed between the center of curvature of the second curved portion and the edge portion of the plate member.

In some implementations, the apparatus includes an actuation member operatively coupled to the needle and being configured to move the needle towards the needle catch. In some implementations, the apparatus includes a needle carrier configured to engage the needle.

According to another aspect, an apparatus includes an elongate member; a needle configured to extend from a portion of the elongate member; and a needle catch configured to receive at least a portion of the needle, the needle catch having a plate member defining an opening, the plate member having an edge portion that is disposed adjacent the opening, the edge portion of the plate member having a curved portion, the curved portion being curved away from the opening defined by the plate member.

In some implementations, the plate member is non-planar. In some implementations, the portion of the plate member that includes the edge portion is non-planar.

In some implementations, the apparatus includes an actuation member operatively coupled to the needle and being configured to move the needle towards the needle catch. In some implementations, apparatus includes a needle carrier configured to engage the needle.

### BRIEF DESCRIPTION OF THE DRAWINGS

FIG. 1 schematically illustrates a medical device according to an implementation.

FIG. 2 is a side view of a medical device according to an implementation.

FIG. 3 is a perspective view of an end portion of the medical device of FIG. 2.

FIG. 4 is a perspective view of a portion of a needle catch of the medical device of FIG. 2.

FIGS. 5 and 6 are top views of the needle catch of the medical device of FIG. 2.

FIG. 7 is a cross-sectional view of the needle catch of the medical device of FIG. 2 taken along line A-A of FIG. 5.

FIG. 8 is a perspective view of an end portion of a medical device according to an implementation.

FIG. 9 is a perspective view of a portion of a needle catch of the medical device of FIG. 8.

FIGS. 10 and 11 are top views of the needle catch of the medical device of FIG. 8.

FIG. 12 is a cross-sectional view of the needle catch of the medical device of FIG. 8 taken along line B-B of FIG. 10.

FIG. 13 is a perspective view of an end portion of a medical device according to an implementation.

FIG. 14 is a perspective view of a portion of a needle catch of the medical device of FIG. 13.

FIGS. 15 and 16 are top views of the needle catch of the medical device of FIG. 13.

FIG. 17 is a cross-sectional view of the needle catch of the medical device of FIG. 13 taken along line C-C of FIG. 15.

FIGS. 18 and 19 are cross-sectional views of the needle catch of the medical device of FIG. 13 taken along line D-D of FIG. 15.

### DETAILED DESCRIPTION

Detailed implementations are disclosed herein. However, it is understood that the disclosed implementations are merely examples, which may be embodied in various forms. Therefore, specific structural and functional details disclosed herein are not to be interpreted as limiting, but merely as a basis for the claims and as a representative basis for teaching one skilled in the art to variously employ the embodiments in virtually any appropriately detailed structure. Further, the terms and phrases used herein are not intended to be limiting, but to provide an understandable description of the present disclosure.

The terms “a” or “an,” as used herein, are defined as one or more than one. The term “another,” as used herein, is defined as at least a second or more. The terms “including” and/or “having”, as used herein, are defined as comprising (i.e., open transition). The term “coupled” or “moveably coupled,” as used herein, is defined as connected, although not necessarily directly and mechanically.

The terms proximal and distal described in relation to various medical devices, apparatuses, and components (as discussed in the subsequent text of the present invention) are referred to with a point of reference. The point of reference, as used in this description, is a perspective of an operator. The operator may be a surgeon, a physician, a nurse, a doctor, a technician, and the like, who may perform the procedure of surgery through the patient's body orifice as described in the present invention. The term proximal refers to an area that is closest to the operator. The term distal refers to an area that is farthest from the operator. The patient can be a male, a female or any other mammal.

The present invention relates to an apparatus, a medical device, or a suturing device. The suturing device may be used for the treatment of a pelvic floor disorder such as genital prolapse, incontinence, cystoceles, rectoceles, and the like. However, the suturing device can be used for any procedure that requires sutures to be thrown or passed through portions of the body remotely from outside the body. The surgical treatment of these disorders requires sutures to be placed through body tissues. These sutures may connect the pelvic organs to the pelvic floor or they can be used to place implants inside a body of a patient.

Various embodiments, aspects, or implementations of the suturing device are described herein. They should be considered as exemplary and should not be used to limit the scope of the invention.

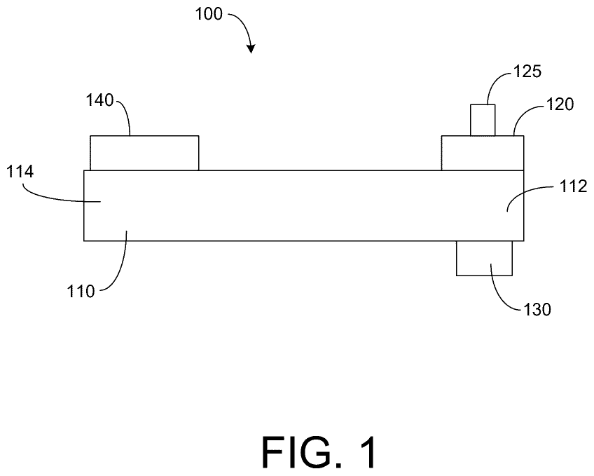

FIG. 1 is a schematic diagram of an apparatus or suturing device 100. The suturing device 100 may be configured to be used to place sutures or to insert bodily implants (not shown) inside a patient's body (e.g., a female patient or a male patient). In some implementations, the suturing device 100 is configured to be used to place sutures into the patient's body by insertion of the suturing device 100 into the patient's body from outside (e.g., insertion via a vaginal incision in the body of the patient, or insertion via a rectal incision in the body of the patient). The suturing device 100 may be used to suture tissues and ligaments together. In some implementations, the suturing device 100 is configured to suture a bodily implant inside the patient's body.

The suturing device 100 includes an elongate body member 110, a needle carrier 120, a needle 125, a needle deployment mechanism 140, and a needle receiving portion or a needle catch 130. The elongate body member 110 includes a distal end portion 112 and a proximal end portion 114. In some implementations, the needle deployment mechanism 140 is disposed at the proximal end portion 114 of the elongate body member 110. In some implementations, the needle deployment mechanism 140 extends from the proximal end portion 114 to the distal end portion 112. In some implementations, the needle deployment mechanism 104 is at least partially disposed within the elongate body member 102. In some implementations, the needle carrier 120 and the needle 125 are disposed at the distal end portion 112 of the elongate body member 110.

The needle deployment mechanism 140 is operatively coupled to the needle carrier 120 which is operatively coupled to the needle 125. The needle deployment mechanism 140 is configured to move the needle carrier 120 and the needle 125 such that the needle 125 may pass through bodily tissue and/or a bodily implant and towards the needle catch 130. For example, in some implementations, a physician may insert the suturing device 100 into the body of the patient such that the distal end portion 112 of the elongate body member 110 is disposed within the body of the patient and the proximal end portion 114 of the elongate body member 110 is disposed outside of the body of the patient. The physician may then actuate or activate the needle deployment mechanism 140 to move the needle carrier 120 and needle 125 through bodily tissue and/or a bodily implant. The needle 125 may be moved towards and be received by the needle catch 130.

In some implementations, a suture or other anchoring member may be coupled to the needle 125. Once the needle is received by the needle catch 130 the suturing device 100 may be removed or withdrawn from the body of the patient. Accordingly, the suture or other anchoring member has been passed through the bodily tissue and/or bodily implant and may be knotted by the physician.

The needle catch 130 is sized and shaped to receive and retain the needle 125. In some implementations, the needle catch 130 is configured to guide the needle 125 to the correct location on the needle catch 130 for receipt and retention of the needle 125.

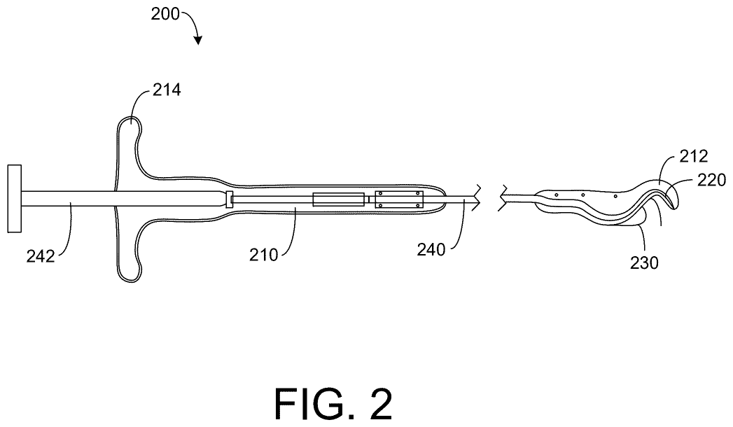

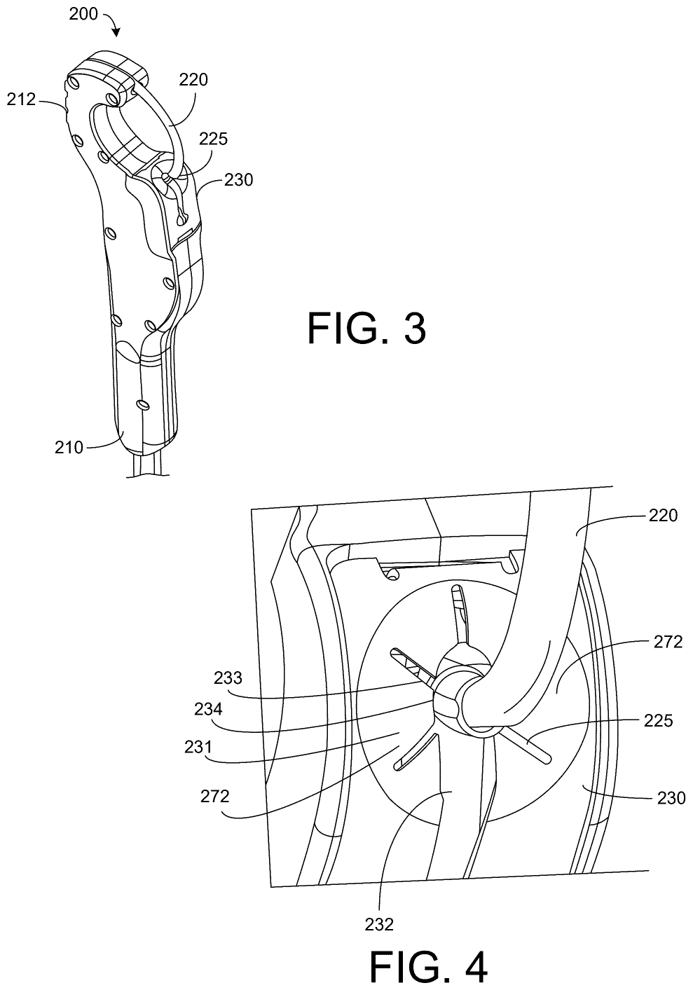

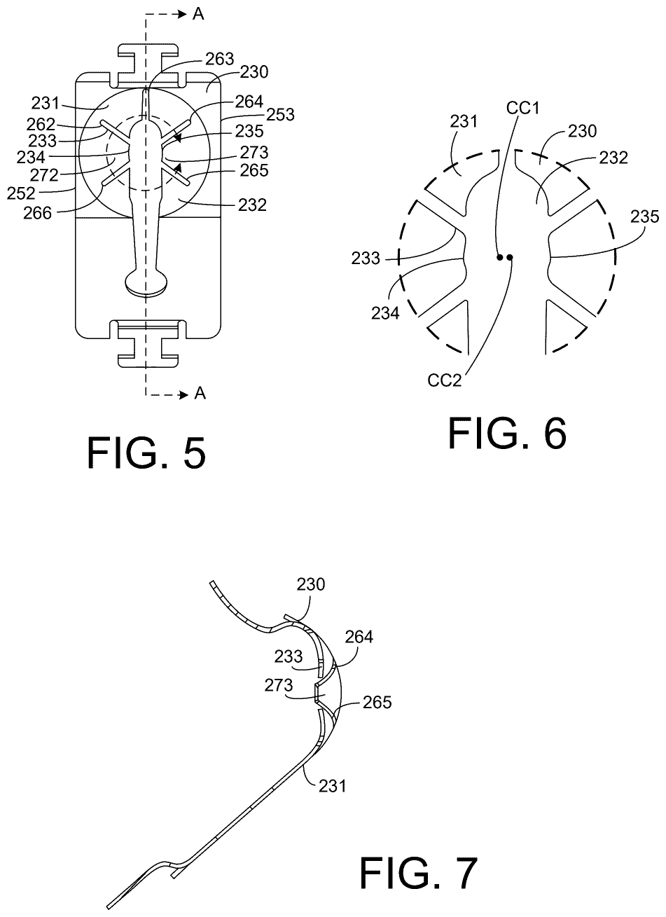

FIG. 2 is a side view of a suturing device 200 according to an implementation. FIG. 3 is a perspective view of a distal portion of the suturing device 200. FIG. 4 is a perspective view of a portion of a needle catch of the suturing device 200. FIGS. 5 and 6 are top views of the needle catch of the suturing device 200. FIG. 7 is a cross-sectional view of the needle catch of the suturing device taken along line A-A of FIG. 5.

The suturing device 200 is configured to be used to place sutures or to insert bodily implants (not shown) inside a patient's body (e.g., a female patient or a male patient). In some implementations, the suturing device 200 is configured to be used to place sutures into the patient's body by insertion of the suturing device 200 into the patient's body from outside (e.g., insertion via a vaginal incision in the body of the patient, or insertion via a rectal incision in the body of the patient). The suturing device 200 may be used to suture tissues and ligaments together. In some implementations, the suturing device 200 is configured to suture a bodily implant inside the patient's body.

The suturing device 200 includes an elongate body member 210, a needle carrier 220, a needle 225, a needle deployment mechanism 240, and a needle receiving portion or a needle catch 230. The elongate body member 210 includes a distal end portion 212 and a proximal end portion 214. In the illustrated implementation, the needle deployment mechanism 240 extends from the proximal end portion 214 to the distal end portion 212. The needle deployment mechanism 240 includes an actuator 242. In the illustrated implementation, the needle carrier 220 and the needle 225 are disposed at the distal end portion 212 of the elongate body member 210.

The needle deployment mechanism 240 includes an actuator 242 and is operatively coupled to the needle carrier 220. The needle carrier 220 is operatively coupled to the needle 225. The needle deployment mechanism 240 is configured to move the needle carrier 220 and the needle 225 such that the needle 225 may pass through bodily tissue and/or a bodily implant and towards the needle catch 230. For example, in some implementations, a physician may insert the suturing device 200 into the body of the patient such that the distal end portion 212 of the elongate body member 210 is disposed within the body of the patient and the proximal end portion 214 of the elongate body member 210 is disposed outside of the body of the patient. The physician may then actuate or activate the needle deployment mechanism 240 (for example, by engaging or moving the actuator 242) to move the needle carrier 220 and needle 225 through bodily tissue and/or a bodily implant. The needle 225 may be moved towards and be received by the needle catch 230. In the illustrated implementation, the needle carrier 220 is non-linear and the needle 225 is linear. In other implementations, the needle carrier may be linear and/or the needle may be non-linear.

A suture S may be coupled to the needle 225. Once the needle 225 is received by the needle catch 230 the suturing device 200 may be removed or withdrawn from the body of the patient. Accordingly, the suture S has been passed through the bodily tissue and/or bodily implant and may be knotted by the physician.

The needle catch 230 is sized and shaped to receive and retain the needle 225. In some implementations, the needle catch 230 is configured to guide the needle 225 to the correct location on the needle catch 230 for receipt and retention of the needle 225.

The needle catch 230 defines an opening 232. Specifically, the needle catch 230 has a plate member 231 that has an edge 233. The edge 233 surrounds and defines the opening 232. The opening 232 is configured to receive at least a portion of the needle 225. In some implementations, the needle catch 230 is configured to help retain the needle 225 within the opening 232 once the needle 225 has been inserted into the opening 232.

As best illustrated in FIGS. 4-6, the edge 233 includes a first curved portion 234. In some implementations, the first curved portion 234 is configured to match or substantially match a curvature of a portion of the needle 225. The first curved portion 234 curves away from the opening 232 and towards a first side 252 of the plate member 231. As illustrated, the first center of curvature CC1 (the point about which the first curved portion 234 curves) of the first curved portion 234 is disposed such that at least a portion of the opening 232 is disposed between the first center of curvature CC1 and the first curved portion 234 of the edge. The first center of curvature CC1 is also disposed such that at least a portion of the opening 232 is disposed between the first center of curvature CC1 and the first side 252 of the plate member 231. In some implementations, the first center of curvature CC1 is disposed within the opening 232 or within the area defined by the opening 232.

The edge 233 includes a second curved portion 235. In some implementations, the second curved portion 235 is configured to match or substantially match a curvature of a portion of the needle 225. The second curved portion 235 curves away from the opening 232 and towards a second side 253 of the plate member 231. As illustrated, the second center of curvature CC2 (the point about which the second curved portion 235 curves) of the second curved portion 235 is disposed such that at least a portion of the opening 232 is disposed between the second center of curvature CC2 and the second curved portion 235 of the edge. The second center of curvature CC2 is also disposed such that at least a portion of the opening 232 is disposed between the second center of curvature CC2 and the second side 253 of the plate member 231. In some implementations, the second center of curvature CC2 is disposed within the opening 232 or within the area defined by the opening 232. In some implementations, the first center of curvature CC1 and the second center of curvature CC2 are disposed at the same location. In other implementations, the first center of curvature CC1 is disposed at a location different than the location of the second center of curvature CC2.

The opening 232 includes slots 262, 263, 264, 265, and 266. In some implementations, the slots 262, 263, 264, 265, and 266 allow a portion or portions of the plate member 231 to flex or bend when a needle 225 is inserted into the opening 232. For example, the portion 272 of the plate member 231 that is disposed proximate to the first curved portion 234 is also disposed between the slot 262 and slot 266. In some implementations, the portion 272 is configured to bend or flex in the direction of the movement of the needle 225 when the needle 225 is inserted into the opening 232. For example, the portion 272 is configured to bend or flex in the direction of the movement of the needle 225 when the needle contacts it and causes it to bend or flex. The portion 272 that has been bent or flexed is configured to return to its non-bent or non-flexed configuration once the needle 225 is not applying pressure to the portion 272. Similarly, the portion 273 of the plate member 231 that is disposed proximate to the second curved portion 235 is also disposed between the slot 264 and slot 265. In some implementations, the portion 273 is configured to bend or flex in the direction of the movement of the needle 225 when the needle 225 is inserted into the opening 232. For example, the portion 273 is configured to bend or flex in the direction of the movement of the needle 225 when the needle contacts it and causes it to bend or flex. The portion 273 that has been bent or flexed is configured to return to its non-bent or non-flexed configuration once the needle 225 is not applying pressure to the portion 273.

In some implementations, the slots have a different shape. For example, in some implementations, some or all of the slots have a tapered shape. In some implementations, the shape of the slots facilitates or increases the flexibility of the plate member. In some implementations, the portions 272 and 273 are configured to bend or flex in a direction different than the direction of movement of the needle. For example, in some implementations, the portions 272 and 273 are configured to bend or flex in a direction orthogonal to the direction of movement of the needle. In some implementations, the portions 272 and 274 are configured to bend or flex in both direction of movement of the needle and in a second direction, such as a direction substantially orthogonal to the movement of the needle.

As best illustrated in FIG. 7, the plate member 231 is curved. In other words, the plate member 231 is non-planar. In some implementations, the plate member 231 is curved such that the needle 225 is guided to the opening 232. In the illustrated implementation, portion 272 and portion 273 are curved in a direction of movement of the needle 225 when the needle 225 is inserted into the opening 232.

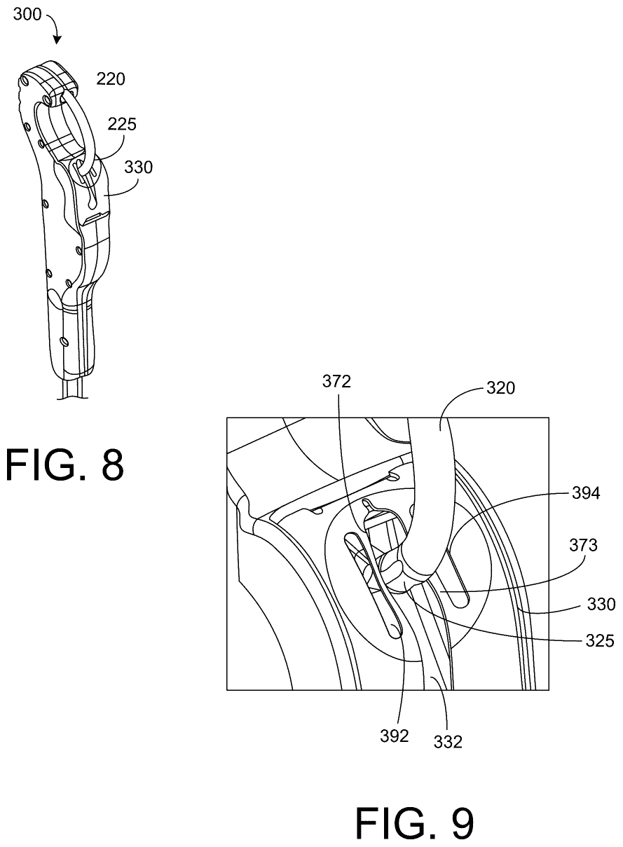

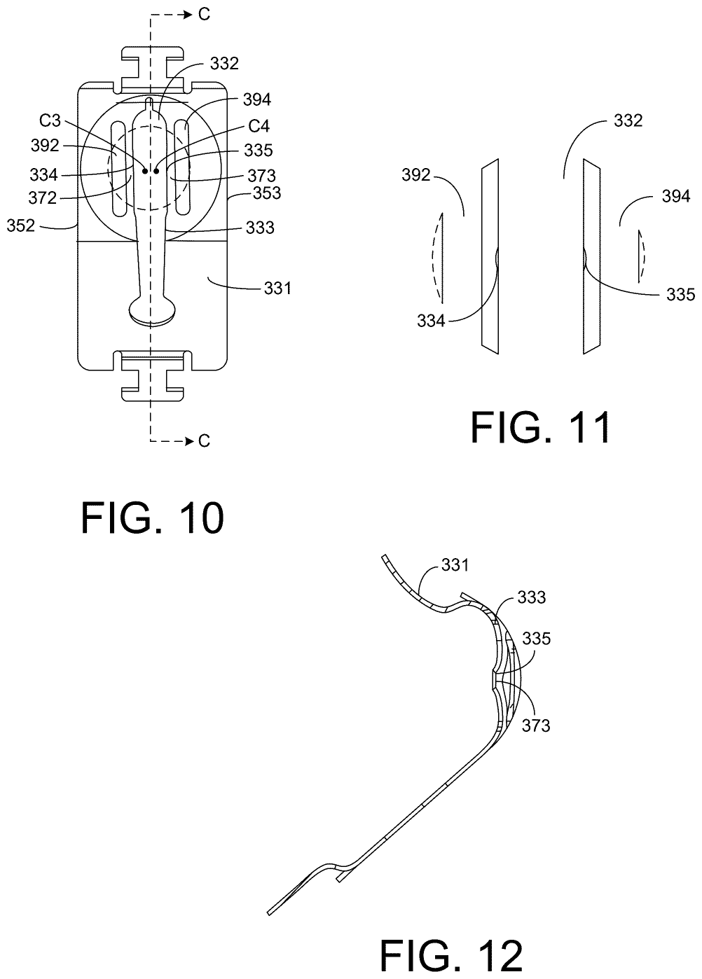

FIG. 8 is a perspective view of an end portion of a medical device 300 according to an implementation. FIG. 9 is a perspective view of a portion of a needle catch 330 of the medical device 300. FIGS. 10 and 11 are top views of the needle catch 330. FIG. 12 is a cross-sectional view of the needle catch 330.

The needle catch 330 is sized and shaped to receive and retain a needle 325. In some implementations, the needle catch 330 is configured to guide the needle 325 to the correct location on the needle catch 330 for receipt and retention of the needle 325.

The needle catch 330 defines an opening 332. Specifically, the needle catch 330 has a plate member 331 that has an edge 333. The edge 333 surrounds and defines the opening 332. The opening 332 is configured to receive at least a portion of the needle 325. In some implementations, the needle catch 330 is configured to help retain the needle 325 within the opening 332 once the needle 325 has been inserted into the opening 332.

As best illustrated in FIGS. 9-11, the edge 333 includes a first curved portion 334. In some implementations, the first curved portion 334 is configured to match or substantially match a curvature of a portion of the needle 325. The first curved portion 334 curves away from the opening 332 and towards a first side 352 of the plate member 331. As illustrated, the first center of curvature CC3 (the point about which the first curved portion 334 curves) of the first curved portion 334 is disposed such that at least a portion of the opening 332 is disposed between the first center of curvature CC3 and the first curved portion 334 of the edge. The first center of curvature CC3 is also disposed such that at least a portion of the opening 332 is disposed between the first center of curvature CC3 and the first side 352 of the plate member 331. In some implementations, the first center of curvature CC3 is disposed within the opening 332 or within the area defined by the opening 332.

The edge 333 includes a second curved portion 335. In some implementations, the second curved portion 335 is configured to match or substantially match a curvature of a portion of the needle 325. The second curved portion 335 curves away from the opening 332 and towards a second side 353 of the plate member 331. As illustrated, the second center of curvature CC4 (the point about which the second curved portion 335 curves) of the second curved portion 335 is disposed such that at least a portion of the opening 332 is disposed between the second center of curvature CC4 and the second curved portion 335 of the edge. The second center of curvature CC4 is also disposed such that at least a portion of the opening 332 is disposed between the second center of curvature CC4 and the second side 353 of the plate member 331. In some implementations, the second center of curvature CC4 is disposed within the opening 332 or within the area defined by the opening 332. In some implementations, the first center of curvature CC3 and the second center of curvature CC4 are disposed at the same location. In other implementations, the first center of curvature CC3 is disposed at a location different than the location of the second center of curvature CC4.

In the illustrated implementation, the needle catch 330 defines a first side opening 392 and a second side opening 394. The opening 332 is disposed between the first side opening 392 and the second side opening 394.

In some implementations, a portion or portions of the plate member 331 are configured to flex or bend when a needle 325 is inserted into the opening 332. For example, the portion 372 of the plate member 331 is disposed proximate to the first curved portion 334. In some implementations, the portion 372 is configured to bend or flex in the direction of the movement of the needle 325 when the needle 325 is inserted into the opening 332. For example, the portion 372 is configured to bend or flex in the direction of the movement of the needle 225 when the needle contacts it and causes it to bend or flex. The portion 372 that has been bent or flexed is configured to return to its non-bent or non-flexed configuration once the needle 225 is not applying pressure to the portion 372. Similarly, the portion 373 of the plate member 331 is disposed proximate to the second curved portion 335. In some implementations, the portion 373 is configured to bend or flex in the direction of the movement of the needle 325 when the needle 325 is inserted into the opening 332. For example, the portion 373 is configured to bend or flex in the direction of the movement of the needle 225 when the needle contacts it and causes it to bend or flex. The portion 373 that has been bent or flexed is configured to return to its non-bent or non-flexed configuration once the needle 225 is not applying pressure to the portion 373.

In some implementations, the portions 372 and 373 are configured to bend or flex in a direction different than the direction of movement of the needle. For example, in some implementations, the portions 372 and 373 are configured to bend or flex in a direction orthogonal to the direction of movement of the needle. In some implementations, the portions 372 and 374 are configured to bend or flex in both direction of movement of the needle and in a second direction, such as a direction substantially orthogonal to the movement of the needle.

As best illustrated in FIG. 12, the plate member 331 is curved. In other words, the plate member 331 is non-planar. In some implementations, the plate member 331 is curved such that the needle 325 is guided to the opening 332. In the illustrated implementation, portion 372 and portion 373 are curved in a direction of movement of the needle 325 when the needle 325 is inserted into the opening 332.

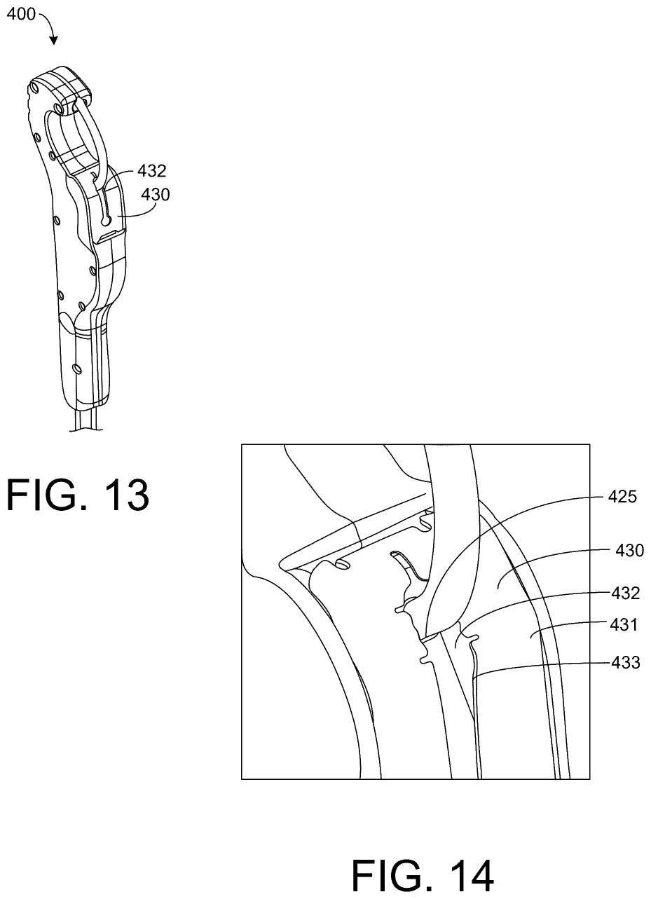

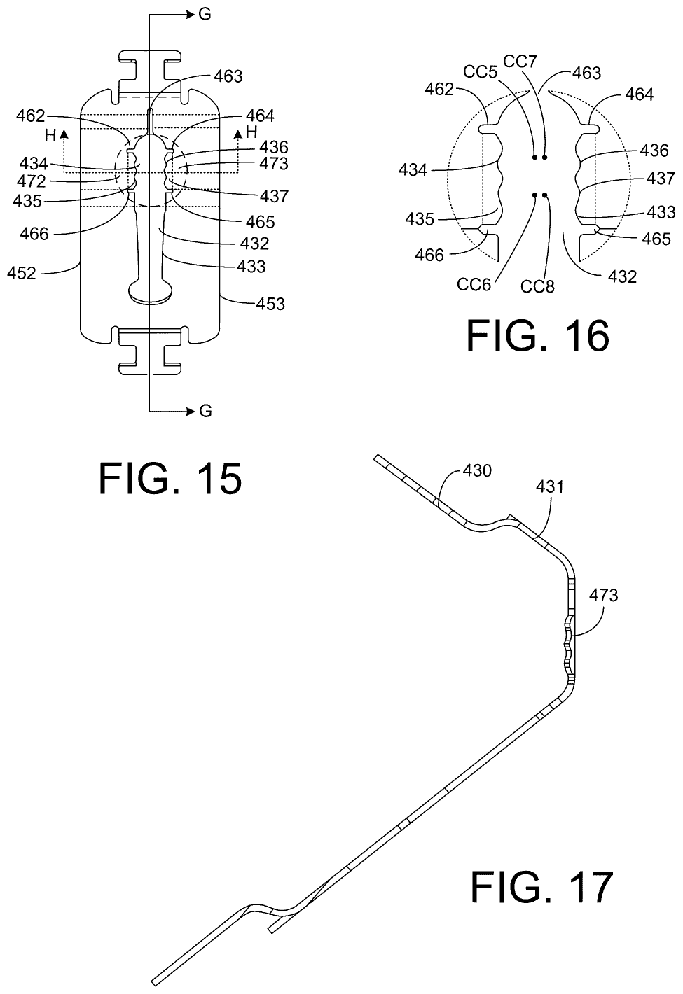

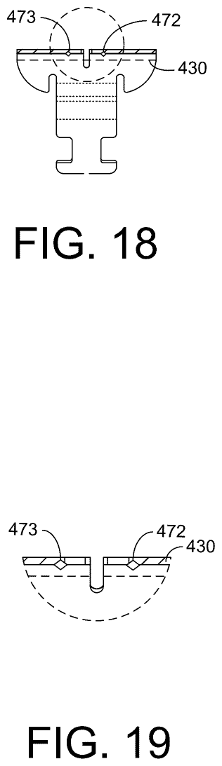

FIG. 13 is a perspective view of an end portion of a medical device 400 according to an implementation. FIG. 14 is a perspective view of a portion of a needle catch 430 of the medical device 400. FIGS. 15 and 16 are top views of the needle catch 430. FIG. 17 is a cross-sectional view of the needle catch 430 taken along line C-C of FIG. 15. FIGS. 18 and 19 are cross-sectional views of the needle catch 430 taken along line D-D of FIG. 15.

The needle catch 430 is sized and shaped to receive and retain the needle 425. In some implementations, the needle catch 430 is configured to guide the needle 425 to the correct location on the needle catch 430 for receipt and retention of the needle 425.

The needle catch 430 defines an opening 432. Specifically, the needle catch 430 has a plate member 431 that has an edge 433. The edge 433 surrounds and defines the opening 432. The opening 432 is configured to receive at least a portion of the needle 425. In some implementations, the needle catch 430 is configured to help retain the needle 425 within the opening 432 once the needle 425 has been inserted into the opening 432.

As best illustrated in FIGS. 14-16, the edge 433 includes a first curved portion 434. The first curved portion 434 curves away from the opening 432 and towards a first side 452 of the plate member 431. As illustrated, the first center of curvature CC5 (the point about which the first curved portion 434 curves) of the first curved portion 434 is disposed such that at least a portion of the opening 432 is disposed between the first center of curvature CC5 and the first curved portion 434 of the edge. The first center of curvature CC5 is also disposed such that at least a portion of the opening 432 is disposed between the first center of curvature CC5 and the first side 452 of the plate member 431. In some implementations, the first center of curvature CC5 is disposed within the opening 432 or within the area defined by the opening 432.

The edge 433 includes a second curved portion 435. The second curved portion 435 curves away from the opening 432 and towards the first side 452 of the plate member 431. As illustrated, the second center of curvature CC6 (the point about which the second curved portion 435 curves) of the second curved portion 435 is disposed such that at least a portion of the opening 432 is disposed between the second center of curvature CC6 and the second curved portion 435 of the edge. The second center of curvature CC6 is also disposed such that at least a portion of the opening 432 is disposed between the second center of curvature CC6 and the first side 452 of the plate member 431. In some implementations, the second center of curvature CC6 is disposed within the opening 432 or within the area defined by the opening 432.

While edge 433 includes two curved portions, in other implementations, the edge may include less than two curved portions (such as one) or may include more than two curved portions.

The edge 433 includes a third curved portion 436. The third curved portion 436 curves away from the opening 432 and towards a second side 453 of the plate member 431. As illustrated, the third center of curvature CC7 (the point about which the third curved portion 436 curves) of the third curved portion 436 is disposed such that at least a portion of the opening 432 is disposed between the third center of curvature CC7 and the third curved portion 436 of the edge. The third center of curvature CC7 is also disposed such that at least a portion of the opening 432 is disposed between the third center of curvature CC7 and the second side 453 of the plate member 431. In some implementations, the third center of curvature CC7 is disposed within the opening 432 or within the area defined by the opening 432.

The edge 433 includes a fourth curved portion 437. The fourth curved portion 437 curves away from the opening 432 and towards the second side 453 of the plate member 431. As illustrated, the fourth center of curvature CC8 (the point about which the fourth curved portion 437 curves) of the fourth curved portion 437 is disposed such that at least a portion of the opening 432 is disposed between the fourth center of curvature CC8 and the fourth curved portion 437 of the edge. The fourth center of curvature CC8 is also disposed such that at least a portion of the opening 432 is disposed between the fourth center of curvature CC8 and the second side 453 of the plate member 431. In some implementations, the fourth center of curvature CC8 is disposed within the opening 432 or within the area defined by the opening 432.

The opening 432 includes slots 462, 463, 464, 465, and 466. In some implementations, the slots 462, 463, 464, 465, and 466 allow a portion or portions of the plate member 431 to flex or bend when a needle 425 is inserted into the opening 432. For example, the portion 472 of the plate member 431 that is disposed proximate to the first curved portion 434 and proximate to the second curved portion 435 is also disposed between the slot 462 and slot 466. In some implementations, the portion 472 is configured to bend or flex in the direction of the movement of the needle 425 when the needle 425 is inserted into the opening 432. For example, the portion 472 is configured to bend or flex in the direction of the movement of the needle 225 when the needle contacts it and causes it to bend or flex. The portion 472 that has been bent or flexed is configured to return to its non-bent or non-flexed configuration once the needle 225 is not applying pressure to the portion 472. Similarly, the portion 473 of the plate member 431 that is disposed proximate to the third curved portion 436 and proximate to the fourth curved portion 437 is also disposed between the slot 464 and slot 465. In some implementations, the portion 473 is configured to bend or flex in the direction of the movement of the needle 425 when the needle 425 is inserted into the opening 432. For example, the portion 473 is configured to bend or flex in the direction of the movement of the needle 225 when the needle contacts it and causes it to bend or flex. The portion 473 that has been bent or flexed is configured to return to its non-bent or non-flexed configuration once the needle 225 is not applying pressure to the portion 473. In some implementations, the portions 472 and 473 are configured to bend or flex in a direction different than the direction of movement of the needle. For example, in some implementations, the portions 472 and 473 are configured to bend or flex in a direction orthogonal to the direction of movement of the needle. In some implementations, the portions 472 and 474 are configured to bend or flex in both direction of movement of the needle and in a second direction, such as a direction substantially orthogonal to the movement of the needle.

As best illustrated in FIGS. 17-19, the plate member 431 is curved. In other words, the plate member 431 is non-planar. In some implementations, the plate member 431 is curved such that the needle 425 is guided to the opening 432. In the illustrated implementation, portion 472 and portion 473 are curved in a direction of movement of the needle 425 when the needle 425 is inserted into the opening 432.

While certain features of the described implementations have been illustrated as described herein, many modifications, substitutions, changes and equivalents will now occur to those skilled in the art. It is, therefore, to be understood that the appended claims are intended to cover all such modifications and changes as fall within the scope of the embodiments.

What is claimed is:

1. An apparatus, comprising:
an elongate member;
a needle configured to extend from a portion of the elongate member; and
a needle catch configured to receive at least a portion of the needle, the needle catch having a plate member defining an opening, the plate member having an edge portion that is disposed adjacent the opening, the edge portion of the plate member having a curved portion, the curved portion being curved about a center of curvature, at least a portion of the opening being disposed between the edge portion and the center of curvature.

2. The apparatus of claim 1, the edge portion of the plate member being a first edge portion of the plate member, wherein the plate member includes a second edge portion that is disposed adjacent the opening, the second edge portion of the plate member having a curved portion.

3. The apparatus of claim 1, the edge portion of the plate member being a first edge portion of the plate member, wherein the plate member includes a second edge portion that is disposed adjacent the opening, the second edge portion of the plate member having a curved portion, the second edge portion being disposed on a first side of the opening defined by the plate member, the second edge portion being disposed on a second side of the opening defined by the plate member.

4. The apparatus of claim 1, the edge portion of the plate member being a first edge portion of the plate member and the center of curvature being a first center of curvature, wherein the plate member includes a second edge portion that is disposed adjacent the opening, the second edge portion of the plate member having a curved portion, the curved portion of the second edge portion being curved about a second center of curvature, at least a portion of the opening being disposed between the second edge portion and the second center of curvature.

5. The apparatus of claim 1, wherein the plate member is non-planar.

6. The apparatus of claim 1, wherein a portion of the plate member disposed adjacent to the edge portion is non-planar.

7. The apparatus of claim 1, wherein the opening defined by the plate member includes a first slot portion and a second slot portion, the edge portion being disposed between the first slot portion and the second slot portion.

8. The apparatus of claim 1, wherein the opening defined by the plate member includes at least 5 slot portions.

9. The apparatus of claim 1, wherein the opening defined by the plate member includes a first slot portion, a second slot portion, a third slot portion, a fourth slot portion, and a fifth slot portion.

10. The apparatus of claim 1, wherein the opening defined by the plate member is a first opening, the plate member defining a second opening.

11. The apparatus of claim 1, wherein the opening defined by the plate member is a first opening, the plate member defining a second opening and a third opening, the first opening being disposed between the second opening and the third opening.

12. The apparatus of claim 1, the curved portion of the edge portion of the plate member being a first curved portion, wherein, the edge portion of the plate member has a second curved portion.

13. The apparatus of claim 1, the curved portion of the edge portion of the plate member being a first curved portion, wherein, the edge portion of the plate member has a second curved portion, the second curved portion of the edge portion having a center of curvature, at least a portion of the opening defined by the plate member being disposed between the center of curvature of the second curved portion and the edge portion of the plate member.

14. The apparatus of claim 1, further comprising:
an actuation member operatively coupled to the needle and being configured to move the needle towards the needle catch.

15. The apparatus of claim 1, further comprising:
a needle carrier configured to engage the needle.

16. An apparatus, comprising:
an elongate member;
a needle configured to extend from a portion of the elongate member; and
a needle catch configured to receive at least a portion of the needle, the needle catch having a plate member defining an opening, the plate member having an edge portion that is disposed adjacent the opening, the edge portion of the plate member having a curved portion, the curved portion being curved away from the opening defined by the plate member.

17. The apparatus of claim 16, wherein the plate member is non-planar.

18. The apparatus of claim 16, wherein the portion of the plate member that includes the edge portion is non-planar.

19. The apparatus of claim 16, further comprising:
an actuation member operatively coupled to the needle and being configured to move the needle towards the needle catch.

20. The apparatus of claim 16, further comprising:
a needle carrier configured to engage the needle.
